(19) United States
(12) Patent Application Publication
(10) Pub. No.: US 2014/0086826 A1
(43) Pub. Date: Mar. 27, 2014

(54) SIN3B COMPLEX INHIBITION FOR PREVENTING HEMATOLOGIC CHEMOTHERAPY- AND RADIOTHERAPY-INDUCED CYTOTOXICITY

(71) Applicant: New York University, New York, NY (US)

(72) Inventors: Gregory David, New York, NY (US); Teresa DiMauro, New York, NY (US)

(73) Assignee: New York University, New York, NY (US)

(21) Appl. No.: 14/033,643

(22) Filed: Sep. 23, 2013

Related U.S. Application Data
(60) Provisional application No. 61/705,376, filed on Sep. 25, 2012.

(51) Int. Cl. A61K 38/43; A61K 45/06

(52) U.S. Cl. CPC A61K 38/43; A61K 45/06
USPC 424/1.11; 424/94.1

(57) ABSTRACT

Methods for inactivating Sin3B and its associated activities to prevent, inhibit or attenuate hematopoietic stem cell exhaustion and chemotherapy- and radiotherapy-induced hematologic cytotoxicity are provided.

### INTRODUCTION

This application claims the benefit of priority of U.S. Provisional Application No. 61/705,376 filed Sep. 25, 2012, the content of which is incorporated herein by reference in its entirety.

This invention was made with government support under contract number 5R01CA148639 awarded by the National Institutes of Health. The government has certain rights in the invention.

### BACKGROUND OF THE INVENTION

Hematopoietic stem cells (HSCs) represent the source of all mature blood cells in adults. As blood cells need continuous replenishment throughout adult life, functional failure in HSCs self-renewal and/or the ability to differentiate can result in bone marrow failure and fatal consequences to the organism. During a normal lifetime, HSC function over the long-term and may not exhibit functional loss under physiological circumstances. However, pathologic environments, such as those triggered by genotoxic stress, limit HSC proliferation and shorten the functional life of HCS. This leads to HSC exhaustion, irreversible exit from the cell cycle and subsequent inability to self-renew. It has been demonstrated that in the bone marrow of patients treated with ionizing radiation or high-dose chemotherapy, and patients affected by myelodysplastic syndromes (MDS), HSC undergo a permanent cell cycle exit reminiscent of cellular senescence, leading to long-term bone marrow failure.

The Sin3 complex is highly conserved throughout evolution, and mammalian cells contain two Sin3 proteins, Sin3A and Sin3B (Ayer, et al. (1995) Cell 80:767-776; Schreiber-Agus, et al. (1995) Cell 80:777-786; Vidal, et al. (1991) Mol. Cell Biol. 11:6306-6316; Wang, et al. (1990) Mol. Cell Biol. 10:5927-5936). Sin3 proteins are non-catalytic scaffold proteins that serve as an evolutionarily conserved component of the histone deacetylase HDAC1/2 transcriptional repression complex. A large and diverse group of sequence-specific transcription factors interact with the ubiquitously expressed Sin3A and/or Sin3B, and these interactions result in transcriptional repression (Silverstein & Ekwall (2005) Curr. Genet. 47:1-17). While most work has focused on its homolog Sin3A and its role as a co-repressor for numerous sequence-specific transcription factors (Silverstein & Ekwall (2005) supra), it has been recently demonstrated that the cellular functions of Sin3A and Sin3B are not redundant (Dannenberg, et al. (2005) Genes Dev. 19:1581-1595; David, et al. (2008) Proc. Natl. Acad. Sci. USA 105:4168-4172). Specifically, it was shown that both Sin3A and Sin3B are essential for embryonic development, but possess distinct properties (Dannenberg, et al. (2005) supra; David, et al. (2008) supra). Sin3A and the associated Sds3 protein are essential for the generation of repressed chromatin structure at pericentric loci, and their genetic inactivation results in aberrant chromosomal segregation and cell death (Dannenberg, et al. (2005) supra; David, et al. (2003) Genes Dev. 17:2396-2405). By contrast, Sin3B is dispensable for cellular viability (David, et al. (2008) supra). Studies have indicated that a Sin3B-containing complex interacts with Rb family tumor suppressor and regulates the transcriptional repression of E2F target genes upon cell cycle withdrawal (Balciunaite, et al. (2005) Mol. Cell Biol. 25:8166-8178; David, et al. (2008) supra; Grandinetti & David (2008) Cell Cycle 7:1550-1554; Rayman, et al. (2002) Genes Dev. 16:933-947). Sin3B knockout mice and cells have been generated and it was demonstrated that Sin3B is essential for cell cycle exit upon quiescence and differentiation, through its ability to tether chromatin repressors on E2F target promoters (David, et al. (2008) supra; Grandinetti & David (2008) supra). These observations were expanded in the context of cellular senescence. Specifically, mouse embryonic fibroblasts genetically inactivated for Sin3B (Sin3B−/− MEFs) were, by contrast to their wild-type counterparts, refractory to replicative senescence and oncogenic Ras-induced senescence, as evidenced by continuous BrdU incorporation and decreased SA-β-gal staining (Grandinetti, et al. (2009) Cancer Res. 69:6430-6437). RasV12 expression leads to the direct recruitment of Sin3B and the Sin3B-dependent recruitment of chromatin repressors at pro-proliferative loci. Consistently, in the absence of Sin3B, E2F target genes fail to be transcriptionally repressed following RasV12 ectopic expression.

Purification of the complex(es) associated with Sin3 proteins identified the components of what has been defined as the core complex including HDAC1/2 (Alland, et al. (2002) Mol. Cell Biol. 22:2743-2750; Doyon, et al. (2006) Mol. Cell 21:51-64; Fleischer, et al. (2003) Mol. Cell Biol. 23:3456-3467; Kuzmichev, et al. (2002) Mol. Cell Biol. 22:835-848; Lai, et al. (2001) Mol. Cell Biol. 21:2918-2932; Skowyra, et al. (2001) J. Biol. Chem. 276:8734-8739). Additional unbiased proteomic approaches in mammalian systems have led to the purification of proteins that associate stably and specifically with Sin3B, but not Sin3A. These include Pf1 (also known as Phf12, likely to be the homolog of yeast Rco1), KDM5A (a histone H3 lysine 4 demethylase, also known as Jarid1A or Rbp2), MrgX, EMSY and GATAD1 (Bartke, et al. (2010) Cell 143:470-484; Hayakawa, et al. (2007) Genes Cells 12:811-826; Malovannaya, et al. (2011) Cell 145:787-799; Vermeulen, et al. (2010) Cell 142:967-980). Notably, these proteins were consistently found to associate with Sin3B in several independent purifications, suggesting they make up the full Sin3B-Pf1 complex.

### SUMMARY OF THE INVENTION

This invention is a method for preventing, attenuating or inhibiting hematopoietic stem cell (HSC) exhaustion by administering to a subject in need thereof (e.g., a subject with cancer, bone marrow failure syndrome or myelodysplastic syndrome) an effective amount of a Sin3B Complex inhibitor. In some embodiments, the inhibitor is administered in combination with a chemotherapy or radiotherapy.

The invention also provides a method for preventing, attenuating or inhibiting chemotherapy- and radiotherapy-induced hematologic cytotoxicity by administering to a subject receiving or about to receive chemotherapy or radiotherapy an effective amount of a Sin3B Complex inhibitor.

In some embodiments of these methods, the protein of the Sin3B Complex is Sin3B, HDAC1, HDAC2, KDM5A, MeCP2, SMRT, Pf1, MrgX, EMSY or GATAD1. In other embodiments, the inhibitor inhibits the expression Sin3B or is a Sin3B scaffold inhibitor. A kit containing an inhibitor of a protein of the Sin3B Complex; and (b) a chemotherapeutic or radiotherapeutic agent is also provided.

### DETAILED DESCRIPTION OF THE INVENTION

Hematopoiesis is a tightly regulated process that involves self-renewal of stem cells, expansion of lineage-committed progenitor populations, and maturation of terminally differentiated cell types. Once established, the hematopoietic system supplies an organism with the various blood cell lineages in a regulated manner. Human HSCs enter the cell cycle on average once every 40 weeks (Catlin et al. (2011) Blood 117:4460-6). They transit through the cell cycle at a slow rate compared with their more differentiated progenitor cells due to an extended passage through the G1 phase (Nygren, et al. (2006) J. Immunol. 177:201-208). Moreover, disruption of HSC quiescence leads to defects in HSC self-renewal and often results in HSC exhaustion, i.e., loss of stem cell function (Orford & Scadden (2008) Nature Rev. Genetics 9:115-128).

It has now been shown that genetic inactivation of Sin3B in the hematopoietic system prevents HSC cell cycle exhaustion in response to different stresses, including gamma-irradiation and genetic inactivation of the polycomb protein Bmi-1. In addition, it has been demonstrated that these effects are cell autonomous, as Sin3B-inactivated HSC are refractory to the detrimental effects of irradiation and Bmi-1 loss. Based, on these observations, a novel molecular pathway has been identified that regulates the homeostatis of the hematopoietic system in response to stress.

Therefore, this invention provides a method for preventing, attenuating or inhibiting HSC cell cycle exhaustion by inhibiting the expression or activity of a protein of the Sin3B Complex. In certain embodiments, the HSC cell cycle exhaustion of the invention is induced by stress, in particular stress induced by chemotherapy or radiotherapy. Therefore, the invention also provides a method for preventing, attenuating or inhibiting HSC cell cycle exhaustion in a subject receiving chemotherapy or radiotherapy and a method for preventing, attenuating or inhibiting hematologic chemotherapy- and radiotherapy-induced cytotoxicity in a subject by inhibiting the expression or activity of a protein of the Sin3B Complex. In this respect, adjuvant therapy with a Sin3B Complex inhibitor can be used to prevent the short-term detrimental effects of irradiation and high dose chemotherapy.

As has been described in the art, “HSC exhaustion” refers to the irreversible exit of HSC from the cell cycle and inability to self-renew. In one embodiment, HSC exhaustion is induced by chemotherapy or radiotherapy. Chemotherapeutic and/or radiotherapeutic treatment of a subject is typically carried out in such fashion that the chemotherapeutic and/or radiotherapeutic agent(s) is applied in several cycles, with one or more therapeutic agents. By way of illustration, the BEAM conditioning regimen, that is mainly used as a preparative treatment for autologous bone marrow transplantation in Hodgkin's lymphoma, and sometimes also in non-Hodgkin's lymphoma includes the administration of BCNU 300 mg/m² on day 1; etoposide 200 mg/m²/day on days 2, 3, 4 and 5; ARA-C 200 mg/m² every 12 hours, on days 2, 3, 4 and 5; and melphalan 140 mg/m² on day 6. For treatment of classic Hodgkin lymphoma, radiation therapy is generally administered in combination with chemotherapy. Doses used in combined modality therapy are 30-36 Gy for bulky disease sites and 20-30 Gy for nonbulky disease sites. When radiation therapy is used alone, doses may range from 30-44 Gy. As another example, the VIP treatment scheme of Ewing sarcoma involves the administration of etoposide 100 mg/m²/day for 3 days, ifosfamide 1000 mg/m²/day for 5 days, cisplatinum 20 mg/m²/day for 5 days, a treatment, which is sometimes alternated with the VAI scheme that includes vincristine 2 mg/day for 3 days, adrimycine 20 mg/m²/day for 3 days and ifosfamide 3 g/m²/day for 3 days. These schemes and others like it used in the treatment of cancer are within the scope of the present invention.

Upon treatment with one or multiple cycles (e.g., with 3 to 4 weeks intermission in between cycles) of the radiotherapeutic and/or chemotherapeutic agent(s), most of the progenitor cells are killed. Therefore, the stem cells proliferate and differentiate in the time between the cycles. As a consequence of this, after several treatment cycles, an exhaustion of the stem cell compartment is often observed in the subject. Inhibiting the expression or activity of a protein of the Sin3B Complex can prevent or attenuate such exhaustion if the inhibitor is administered before, during and/or after a treatment cycle. It is preferred to administer the Sin3B inhibitor at least 3 to hours before the first treatment cycle, during the cycle, and preferably, up to 1 to 2 weeks after the cycle.

Patients may be human or non-human and may include, for example, animal strains or species used as “model systems” for research purposes. A patient may include either adults or juveniles (e.g., children). The term “patient,” further refers to any living organism, preferably a mammal (e.g., human or non-human) that may benefit from the administration of compositions contemplated herein.

In specific embodiments, subjects receiving a Sin3B inhibitor will exhibit a reduction or attenuation of HSC exhaustion by at least about 5%, 10%, 15%, 20%, 25%, 30%, 35%, 40%, 45%, 50%, 55%, 60%, 65%, 80%, 85%, 90%, 95%, or 100% as compared to a subject not receiving such treatment. The level of HSC exhaustion in a subject can be assessed by methods well known in the art. For example, human hematopoietic progenitor cells and hematopoietic stem cells are characterized by a high expression of CD34 and the absence of CD38 expression (CD34⁺⁺CD38⁻ cells). Upon differentiation and lineage commitment, the expression of CD38 increases while the expression of CD34 decreases (CD34⁺CD38⁺ cells) (Terstappen, et al. (1991) Blood 77:1218; Huang & Terstappen (1992) Nature 360:745). To study the effects of an inhibitor of the Sin3B Complex on the development of HSC, primary cultures of CD34⁺⁺CD38⁻ cells can be contacted with the inhibitor in the presence of a radiotherapeutic and/or chemotherapeutic and the output of secondary colony-forming cells (CFC) can be analyzed.

The prevention, attenuation or inhibition of HSC cell cycle exhaustion in a subject finds application in preventing, attenuating or inhibiting hematologic chemotherapy- and radiotherapy-induced cytotoxicity; long-term bone marrow failure, as well as in the treatment of bone marrow failure syndromes and related diseases (e.g., myelodysplastic syndromes). The bone marrow failure syndromes include a group of disorders than can be either inherited or acquired. In particular embodiments, the bone marrow failure syndrome being treated is acquired, e.g., as a result of toxicity by chemicals (e.g., benzene, busulfan or chloramphenicol) or radiation, as well as association with viruses (e.g., human parvovirus or human cytomegalovirus) and other infectious agents. The most common cause of acquired bone marrow failure is aplastic anemia. See Chen (2005) Clin. Med. Res. 3:102-8.

Sin3A/B are large multidomain proteins that contain four paired amphipathic α-helices (PAH) known as PAH domains, a central HDAC interaction domain (HID) to which almost all of the core corepressor components bind, and a C-terminal highly conserved region (HCR). As well as serving as a bridge between transcription factors and histone deacetylation (HDAC) and histone demethylation activities (e.g., KDM5A or Jarid1A), the Sin3 Complex has also been shown to interact with the methylated DNA binding protein MeCP2 and the HDAC-associated corepressor silencing mediator of retinoic acid and thyroid hormone receptor (SMRT) (Silverstein & Ekwall (2005) Curr. Genet. 47:1-17), as well as Pf1, MrgX, EMSY and GATAD1 (Bartke, et al. (2010) supra; Hayakawa, et al. (2007) supra; Malovannaya, et al. (2011) supra; Vermeulen, et al. (2010) supra). Therefore, a “Sin3B Complex,” as used herein, is intended to include one or more of the following proteins: Sin3B, HDAC1, HDAC2, KDM5A, MeCP2, SMRT, Pf1, MrgX, EMSY and GATAD1. Any agent that inhibits the ability of one or more proteins to interact with Sin3B (referred to herein as a Sin3B scaffold inhibitor), or alternatively inhibits the associated enzymatic activities is considered a Sin3B Complex inhibitor that inhibits the activity of a protein of the Sin3B Complex.

Agents of use in the methods of the invention can inhibit either the expression or activity of a protein of the Sin3B Complex. In one embodiment, the Sin3B Complex inhibitor inhibits the activity of a protein of the Sin3B Complex. Agents of use in accordance with this embodiment of the invention typically include protein- or peptide-based inhibitors or small organic molecules. For the purposes of the present invention, a “protein-based” or “peptide-based” inhibitor is an inhibitor composed of two or more amino acid residues covalently attached by peptide bonds, which may be further modified to include organic and/or inorganic groups. Protein-based or peptide-based inhibitors include Sin3B binding proteins or peptides that bind to Sin3B thereby blocking the interaction between Sin3B and one or more chromatin modifying enzymes. One example of a suitable peptide-based inhibitor is a decoy peptide, e.g., a Sin3 interaction domain (SID) peptide, which interferes with recruitment of MAD and HDAC1 by Sin3B (Farias, et al. (2010) Proc. Natl. Acad. Sci. USA 107:11811-6; US 2011/0003753). SID decoy peptides of use in this invention include the peptides VRMNIQMLLEAADYLERRER (SEQ ID NO:1), MNIQMLLEAADYLE (SEQ ID NO:2), MNIQMLLEAPDYLE (SEQ ID NO:3), or MNIQMPLEAPDYLE (SEQ ID NO:4), which can optionally contain a leader sequence (YGRKKRRQGGG, SEQ ID NO:5) corresponding to the human immunodeficiency virus type 1 Tat arginine-rich RNA-binding motif (ARM), which has been mutated (RRR>GGG) to improve nuclear entry.

A variety of small organic molecules also find use in the methods of this invention. In particular embodiments, the small organic molecules inhibit the activity of enzymes associated with Sin3B. These molecules include histone deacetylation inhibitors such as romidepsin, hydroxamic acids (e.g., trichostatin A, vorinostat, belinostat, LAQ824 and panobinostat), cyclic tetrapeptides (e.g., trapoxin B), benzamides (e.g., entinostat, CI994 and mocetinostat), electrophilic ketones, and aliphatic compounds (e.g., phenylbutyrate and valproic acid); or histone demethylation inhibitors such as 8-hydroxyquinolines, flavanoids, catechols, and N-oxalyl glycine derivatives. See, King, et al. (2010) PLoS One 5:e15535; Lohse, et al. (2011) Bioorgan. Med. Chem. 19:3625-36.

In another embodiment, the Sin3B Complex inhibitor inhibits the expression of a protein of the Sin3B Complex. Agents of use in accordance with this embodiment of the invention are typically oligonucleotide-based inhibitors composed of two or more nucleotides (RNA or DNA) and/or peptide-nucleic acids that inhibit the expression and/or activity of Sin3B. Preferably, oligonucleotides-based inhibitors decrease the level of expression of an endogenous gene (e.g., by decreasing transcription of the Sin3B gene). In particular embodiments, oligonucleotides-based inhibitors include antisense oligonucleotides (ODNs), interfering RNA, ribozymes and DNAzymes as sequence-specific inhibitors of gene expression.

Antisense oligonucleotides can be complementary to the entire coding region of protein of interest, but more preferably is an oligonucleotide which is antisense to only a portion of the coding or noncoding region of a protein of interest, e.g., the Sin3B disclosed in GENBANK Accession No. NM_{—}015260. For example, the antisense oligonucleotide can be complementary to the region surrounding the translation start site of the mRNA. An antisense oligonucleotide can be, for example, about 5, 10, 15, 20, 25, 30, 35, 40, 45 or 50 nucleotides in length. An antisense nucleic acid can be constructed using chemical synthesis and enzymatic ligation reactions using procedures known in the art. For example, an antisense oligonucleotide can be chemically synthesized using naturally occurring nucleotides or variously modified nucleotides designed to increase the biological stability of the molecules or to increase the physical stability of the duplex formed between the antisense and sense nucleic acids, e.g., phosphorothioate derivatives and acridine substituted nucleotides can be used. Alternatively, the antisense nucleic acid can be produced biologically using an expression vector into which a nucleic acid has been subcloned in an antisense orientation.

RNA-interfering inhibitors are small nucleic acid molecules that downregulate, inhibit, or reduce the expression of Sin3B. Non-limiting examples of such nucleic acid molecules include short interfering nucleic acid (“siNA”), short interfering RNA (“siRNA”), double stranded RNA (“dsRNA”), microRNA (“miRNA”), and short hairpin RNA (“shRNA”). Techniques for making these nucleic acid molecules are disclosed, for example, in U.S. Pat. Nos. 5,514,567; 5,561,222; 6,506,559; 7,022,828; 7,078,196; 7,176,304; 7,282,564; and 7,294,504; which are incorporated herein by reference in their entirety. Exemplary siRNA molecules include GCAAAGCGGUCCCUGUUUAUU (SEQ ID NO:6) and GGCAAUGGGUCGUGCGAGAUU (SEQ ID NO:7), which have been shown to inhibit Sin3B expression (van Oevelen, et al. (2008) Mol. Cell 32:359-370).

The inhibitory agents described herein can also be administered as an adjuvant to a cancer therapy. In this respect, the agent may be administered in conjunction with conventional chemotherapy and/or radiotherapy. Accordingly, the invention also provides a kit containing the Sin3B inhibitory agent and one or more chemotherapeutic and/or radiotherapeutic agents.

The Sin3B Complex inhibitors described herein can be administered alone or in combination with a physiologically or pharmaceutically acceptable carrier, excipient, or stabilizer. The term “pharmaceutically acceptable” means a non-toxic material that does not interfere with the effectiveness of the biological activity of the active ingredients. The term “pharmaceutically-acceptable carrier” means one or more compatible solid or liquid fillers, diluents or encapsulating substances which are suitable for administration to a human or other vertebrate animal. The term “carrier” refers to an organic or inorganic ingredient, natural or synthetic, with which the active ingredient is combined to facilitate the application.

Pharmaceutical compositions may be formulated in a conventional manner using one or more physiologically acceptable carriers including excipients and auxiliaries which facilitate processing of the active compounds into preparations which can be used pharmaceutically. Proper formulation is dependent upon the route of administration chosen. In the preferred embodiment, administration is by injection. Typical formulations for injection include a carrier such as sterile saline or a phosphate buffered saline. Viscosity modifying agents and preservatives are also frequently added.

Optional pharmaceutically acceptable excipients especially for enteral, topical and mucosal administration, include, but are not limited to, diluents, binders, lubricants, disintegrants, colorants, stabilizers, and surfactants. Diluents, also referred to as “fillers,” are typically necessary to increase the bulk of a solid dosage form so that a practical size is provided for compression of tablets or formation of beads and granules. Binders are used to impart cohesive qualities to a solid dosage formulation, and thus ensure that a tablet or bead or granule remains intact after the formation of the dosage forms. Lubricants are used to facilitate tablet manufacture. Disintegrants are used to facilitate dosage form disintegration or “breakup” after administration, and generally include, but are not limited to, starch, sodium starch glycolate, alginine, gums or cross linked polymers, such as cross-linked PVP. Stabilizers are used to inhibit or retard decomposition reactions which include, by way of example, oxidative reactions. Surfactants may be anionic, cationic, amphoteric or nonionic surface active agents. If desired, the compositions may also contain minor amount of nontoxic auxiliary substances such as wetting or emulsifying agents, dyes, pH buffering agents, or preservatives.

Administration of an inhibitory agent of the invention can be by various routes including, but not limited to, intradermal, intramuscular, intraperitoneal, intravenous, subcutaneous, oral, and/or gastric routes. The outcome of the therapeutic and prophylactic methods disclosed herein is to at least produce in a subject a healthful benefit, which includes, but is not limited to, prolonging the lifespan of a subject, delaying the onset of one or more symptoms of hematologic cytotoxicity or bone marrow failure.

The actual dosage amount of an inhibitory agent of this invention may be determined by physical and physiological factors such as age, sex, body weight, severity of the disease, previous or concurrent therapeutic interventions, idiopathy of the patient and on the route of administration. These factors may be determined by a skilled artisan. The practitioner responsible for administration will typically determine the concentration of active ingredient(s) in a composition and appropriate dose(s) for the individual subject. The dosage may be adjusted by the individual physician in the event of any complication.

Single or multiple doses of the inhibitory agent are contemplated. Desired time intervals for delivery of multiple doses can be determined by one of ordinary skill in the art employing no more than routine experimentation. As an example, subjects may be administered two doses daily at approximately 12 hour intervals. In some embodiments, the inhibitory agent is administered once a day.

The inhibitory agent may be administered on a routine schedule. As used herein a routine schedule refers to a predetermined designated period of time, which may or may not be coordinated with other treatments (e.g., chemotherapeutic or radiotherapeutic treatments). The routine schedule may encompass periods of time which are identical or which differ in length, as long as the schedule is predetermined. For instance, the routine schedule may involve administration twice a day, every day, every two days, every three days, every four days, every five days, every six days, a weekly basis, a monthly basis or any set number of days or weeks there-between. Alternatively, the predetermined routine schedule may involve administration on a twice daily basis for the first week, followed by a daily basis for several months, etc.

The invention will be further described in the following examples, which do not limit the scope of the invention described in the claims.

### EXAMPLE 1

### Materials and Methods

Cellular Analysis. Mouse Embryonic Fibroblasts (MEF) were generated from E13.5 day embryos. MEFs were grown in DMEM supplemented with 10% Fetal Bovine Serum and penicillin/streptomycin. MEFs were cultured at 37° C. with 5% CO₂, 6% O₂, and 100% humidity. MEFs were infected no later than passage 2. HEK293T Cells were grown in DMEM supplemented with 10% Newborn Calf Serum and penicillin/streptomycin. HEK293T Cells were maintained at 37° C. with 5% CO₂ with 100% humidity. Retroviral and lentiviral infection, BrdU, and SA-B-Gal staining were conducted in accordance with known methods (Grandinetti, et al. (2009) Cancer Res. 69:6430-37).

Chromatin Immunoprecipitation (ChIP). ChIP assays were performed in accordance with known methods (Jelinic, et al. (2011) Mol. Cell. Biol. 31:54-62.). Antibodies used for ChIP were as follows: H3 (Abcam ab-1791), H3K4me3 (Millipore 04-745), Bmi-1 (Millipore 17-664). IgG was used as a control.

Mice. All animal experiments were done in accordance with the guidelines of the National Institutes of Health (NIH) and were approved by the NYU School of Medicine Institutional Animal Care and Use Committee. Animals were housed in a barrier animal facility. Bmi-1 deleted mice have been described in the art (van der Lugt, et al. (1994) Genes Dev. 8:757-69). Sin3B^{L/L} Bmi-1^{+/-} Mx-Cre^{+/-} mice of mixed background were crossed with Sin3B^{L/L} Bmi-1^{+/-} mice to generate Sin3B^{L/L}Bmi-1^{+/+}, Sin3B^{L/L}Bmi-1^{+/+}MxCre^{+/-}, Sin3B^{L/L}Bmi-1^{-/-}, and Sin3B″Bmi-1^{-/-}MxCre^{+/-} mice. Mice were injected with 200 μg of poly:IC every other day for one week beginning at weaning age. All mice were sacrificed and analyzed 2 weeks after poly:IC deletion.

Flow Cytometric Analysis and Cell Sorting. Single cell suspensions were derived from bone marrow (femur and tibia) and thymus from adult (>4 week) mice and stained with conjugated anti-mouse antibodies. For analysis of B cell progenitors, B220-APC/Cy7 (BioLegend), IgM PE/Cy7 (eBioscience), CD43 FITC and CD25 APC (BD Biosciences) were used. For analysis of developing T cells, thymocytes were stained with anti-CD8 PE/CY7 (eBioscience), CD4 PE, CD44 Pacific Blue (BioLegend) and CD25 APC (BD Biosciences). Stained cells were quantified using a BD FORTESSA analyzer.

Colony Forming Assay. Pre-B colony-plating assays were performed in methylcellulose-based medium, M3630 (Stem Cell Technologies), with 3×10⁵ bone marrow cells plated in duplicate and scored for colony formation after 8 days.

Intracellular ROS Analysis. B cell progenitors were incubated with 5 μM DCFDA (Invitrogen) for 30 minutes at 37° C. followed immediately by analysis with a BD FORTESSA analyzer.

RNA and Quanititative Reverse Transcription-PCR (qRT-PCR). RNA extraction and cDNA synthesis were performed in accordance with known methods (Grandinetti, et al. (2009). qRT-PCR was performed using the Bio-Rad 2X SYBR Green master mix on a Bio-Rad ICYCLER. Results are reported as relative to β2-microglobulin.

Western Blot Analysis. Cells were lysed in RIPA buffer supplemented with protease (complete and PMSF) and phosphatase inhibitor (NaVO₄ and NaF), allowed to incubate for 30 minutes, and centrifuged at full speed for 30 minutes. Protein was quantified using the Bradford assay and separated with a SDS-PAGE gel. Primary antibodies used were: Sin3B (Santa Cruz Biotechnology AK-12), Sin3A (Santa Cruz Biotechnology A20), p19^{ARF} (Abeam ab80-25), p16I^{NK4A} (Santa Cruz Biotechnology), p-ERK (Cell Signaling 4377), H-Ras (Santa Cruz Biotechnology Sc-520), alpha-tubulin (Sigma T9026), and Bmi-1 (Millipore 05-637).

### EXAMPLE 2

### Bmi-1 Directly Represses Sin3B Expression in Proliferating Cells

It has been demonstrated that Sin3B transcript and protein levels are upregulated in primary cells subjected to oncogenic stress (Grandinetti, et al. (2009) supra). In addition, the Ink4a/Arf locus is transcriptionally repressed in proliferating cells, but becomes actively transcribed following oncogene activation. This repression is mediated in primary fibroblasts by the PcG protein Bmi-1 (Bracken, et al. (2007) Genes Dev. 21:525-530; Jacobs, et al. (1999) Nature 397:164-168). Therefore, it was determined whether Bmi-1 regulates Sin3B expression. Indeed, ectopic expression of Bmi-1 strongly decreased both Sin3B transcript and protein levels in primary MEFs. Importantly, Bmi-1 driven repression of Sin3B was specific, as the levels of the closely related Sin3A protein were not affected by Bmi-1 ectopic expression. Conversely, shRNA-mediated knockdown of Bmi-1 resulted in a significant increase of Sin3B mRNA and protein levels. As Bmi-1 has been reported to prevent the occurrence of senescence, it remained possible that the Bmi-l-dependent modulation of Sin3B was merely due to senescence. However, this was highly unlikely, as Bmi-1 overexpression still resulted in a downregulation of Sin3B in immortalized MEFs (NIH3T3), while Bmi-1 knockdown led to an increase in Sin3B transcript levels in these cells. These results indicate that Sin3B is a bona fide downstream target of the Polycomb protein Bmi-1.

As observed for oncogenic Ras, it was demonstrated that constitutive BRaf activation induces Sin3B expression. To explore the possibility that the Sin3B locus is a direct target of Bmi-1, the tamoxifen-inducible BRaf^{V600E-ERT1} chimeric protein was employed. In early passage MEFs expressing BRaf^{V600E-ERT1}, tamoxifen administration resulted in activation of the BRaf pathway, as evidenced by the accumulation of phosphorylated ERK1/2. More specifically, induction of the BRaf pathway led to the transcriptional activation of Sin3B, p16^{INK4A}, and p19^{ARF}. To examine the molecular mechanisms underlying Bmi-1 modulation of Sin3B expression, ChIP experiments on primary MEFs were performed, before or after BRaf^{V600E} activation. In accordance with previous reports (Bracken, et al. (2007) supra), a decrease in Bmi-1 enrichment at the p16^{INK4A} and p19^{ARF} promoters was observed in senescent cells. Prior to BRaf^{V600E} activation, Bmi-1 was found enriched at the Sin3B transcription start site (TSS) and to a lesser extent at 1 kb downstream of the TSS (+1 kb), compared to a control locus (gapdh). Following BRaf^{V600E} activation, Bmi-1 was released from the Sin3B locus, coinciding with an increase in histone H3 lysine 4 trimethylation (H3K4me3), a well-documented mark of active chromatin. Together, these experiments demonstrate that the repressor Bmi-1 is tethered to the Sin3B locus in proliferating cells and released upon oncogenic stress, allowing its transcription.

### EXAMPLE 3

### Sin3B Null Cells are Refractory to Bmi-1 Knockdown-Induced Cellular Senescence

Bmi-1 depletion in primary fibroblasts leads to cellular senescence (Jacobs, et al. (1999) Nature 397:164-168). Based on the demonstration that Sin3B is a direct target of Bmi-1 repression, and that Sin3B promotes replicative and oncogene-induced senescence (Grandinetti, et al. (2009) supra), it was determined whether Sin3B upregulation contributed to the cellular senescence elicited upon a decrease in Bmi-1 levels. shRNA-mediated knockdown of Bmi-1 in early passage Sin3B^{+/+} and Sin3B^{-/-} MEFs resulted in a 70% reduction in Bmi-1 transcript levels. Further, Bmi-1 depletion correlated with an increase in Sin3B mRNA and protein levels only in wild-type MEFs, while an increase in p16^{INK4A} p19^{+/+} mRNA abundance was detected upon Bmi-1 knockdown in both Sin3B^{+/+} and Sin3B^{-/-} MEFs. Moreover, decreasing Bmi-1 levels in wild-type MEFs resulted in a significant reduction in BrdU incorporation and an increase in SA-β-Gal positivity, indicative of senescence. Strikingly, genetic inactivation of Sin3B fully rescued the cell cycle withdrawal and premature entry into senescence elicited by Bmi-1 knockdown. Sin3B inactivation prevented senescence elicited by Bmi-1 depletion despite the upregulation of p16^{INK4A} and p19_{ARF}, indicating that the products of the Ink4a/Arf locus may be unable to modulate cell cycle exit in the absence of Sin3B. Indeed, similar to what was observed upon Bmi-1 knockdown, Sin3B is required for cell cycle withdrawal and entry into cellular senescence induced by p16^{INK4A} and p19^{ARF} overexpression. Together, these results demonstrate that Sin3B is an essential regulator of the cell cycle withdrawal and entry into senescence elicited upon Bmi-1 depletion.

### EXAMPLE 4

### Genetic Inactivation of Sin3B Partially Rescues the Hematopoietic Defects Elicited Upon Bmi-1 Loss

Genetic inactivation of Bmi-1 in the mouse leads to significant perturbations of the hematopoietic compartment (van der Lugt, et al. (1994) supra). To assess the contribution of Sin3B to the phenotypes elicited by Bmi-1 depletion in a physiologically relevant system, compound mice harboring a conditional Sin3B allele along with the polyinosinic-polycytidylic acid (poly(I:C))-inducible Mx-Cre transgene were generated in a Bmi-1^{-/-} background. At three weeks of age, poly(I:C) was administered, resulting in the rapid and efficient Cre-mediated deletion of Sin3B in the hematopoietic compartment. Two weeks after poly(I:C) administration, the bone marrow and thymus of these mice were collected for analysis. Bone marrow cells from Bmi-1^{-/-} mice displayed increased expression for Sin3B, p16^{INK4A}_{INK4A}, and p19^{ARF} consistent with what was observed in fibroblasts. Bmi-1 inactivation leads to a block in lymphoid development of both T and B cells at the CD4 . . . CD8 . . . double negative (DN) and pro-B stages, respectively (Miyazaki, et al. (2008) Immunity 28:231-245; Raaphorst, et al. (2001) Trends Immunol. 22:682-690). As expected, Bmi-1^{-/-} mice exhibited defective T cell development as evidenced by a decrease in the frequency of CD4⁺CD8⁺ double positive (DP) thymocytes. Acute somatic deletion of Sin3B restored the frequency of thymocytes. Of note, genetic inactivation of Sin3B also released the differentiation block of thymic T cells in Bmi-1^{-/-} mice. Genetic inactivation of Bmi-1 also leads to a striking depletion of bone marrow B cells. Similar to what was observed in T cells, Sin3B deletion partially restored the frequency of B cells in the bone marrow of Bmi-1^{-/-} mice.

Subsequently, it was determined whether Sin3B promoted the hematopoietic defects elicited by Bmi-1 loss in a cell autonomous manner. Bone marrow mononuclear cells (BMMCs) were plated in methocellulose supplemented with IL-7 to induce pre-B cell colonies. This analysis showed that BMMCs were unresponsive to IL-7 and did not form colonies. Somatic Sin3B deletion partially restored the ability of Bmi-1^{-/-} BMMCs to form colonies in response to IL-stimulation, indicating a cell autonomous function of Sin3B in mediating the effects of Bmi-1 loss in the B cell lineage. It has been demonstrated that the accumulation of reactive oxygen species (ROS) was responsible for the perturbed hematopoietic development resulting from Bmi-1 depletion (Liu, et al. (2009) Nature 459:387-392). It has now been shown that upon stress, Sin3B modulates the transcription of nuclear encoded mitochondrial genes. Therefore it was posited that, through its ability to modulate mitochondrial metabolism, Sin3B contributed to the accumulation of ROS in Bmi-1^{-/-} hematopoietic cells. Consistent with the defective B cell development resulting from Bmi-1 inactivation, a significant accumulation of ROS was detected in Bmi-1^{-/-} pre- and immature B cells. In both cell types, the accumulation of ROS elicited by loss of Bmi-1 was almost completely dependent on the presence of Sin3B, as simultaneous inactivation of Sin3B restored ROS levels close to those observed in wild-type cells. These results identify Sin3B as a novel and critical regulator of the developmental defects elicited by Bmi-1 loss in the lymphoid lineage.

Overall, the results presented herein demonstrate that Sin3B is required for both the cellular senescence and hematopoietic defects elicited by Bmi-1 inactivation. Senescence is a cell cycle exit mechanism that prevents the propagation of damaged DNA (Campisi & d'Adda di Fagagna, (2007) Nat. Rev. Mol. Cell Biol. 8:729-740). While the physiological relevance of cellular senescence in preneoplastic lesions is well established, hallmarks of cellular senescence have also been observed in hematopoietic cells following anti-cancer chemotherapy treatment and therapeutic irradiation (Wang, et al. (2009) Exp. Hematol. 37:1310-1317; Wang, et al. (2009) Blood 107:358-366). In this regard, it is striking to note that the ROS accumulation detected in Bmi-1^{-/-} hematopoietic cells is dependent on the presence of Sin3B. ROS accumulation has been shown to contribute to cellular senescence in both serial passaging and oncogene activation (Irani, et al. (1997) Science 275:1649-1652; Lee, et al. (1999) J. Biol. Chem. 274:7936-7940). Therefore, it is posited that a Bmi-1-Sin3B pathway modulates ROS levels, ultimately regulating cellular senescence and lymphoid development. In this respect, it has now been shown that oncogenic stress leads to a Sin3B-dependent modulation of mitochondrial gene expression, correlating with ROS accumulation.

What is claimed is:

1. A method for preventing, attenuating or inhibiting hematopoietic stem cell (HSC) exhaustion comprising administering to a subject in need thereof an effective amount of a Sin3B Complex inhibitor thereby preventing, attenuating or inhibiting HSC exhaustion.

2. The method of claim 1, wherein the Sin3B Complex comprises Sin3B, HDAC1, HDAC2, KDM5A, MeCP2, SMRT, Pf1, MrgX, EMSY or GATAD1.

3. The method of claim 1, wherein the Sin3B Complex inhibitor inhibits the expression Sin3B.

4. The method of claim 1, wherein the Sin3B Complex inhibitor is a Sin3B scaffold inhibitor.

5. The method of claim 1, wherein the subject has cancer.

6. The method of claim 5, wherein the subject will be or has received chemotherapy or radiotherapy.

7. The method of claim 1, wherein the subject has bone marrow failure syndrome or myelodysplastic syndrome.

8. A method for preventing, attenuating or inhibiting chemotherapy- and radiotherapy-induced hematologic cytotoxicity comprising administering to a subject receiving or about to receive chemotherapy or radiotherapy an effective amount of a Sin3B Complex inhibitor thereby preventing, attenuating or inhibiting chemotherapy- and radiotherapy-induced hematologic cytotoxicity in the subject.

9. The method of claim 8, wherein the Sin3B Complex comprises Sin3B, HDAC1, HDAC2, KDM5A, MeCP2, SMRT, Pf1, MrgX, EMSY or GATAD1.

10. The method of claim 8, wherein the Sin3B Complex inhibitor inhibits the expression Sin3B.

11. The method of claim 8, wherein the Sin3B Complex inhibitor is a Sin3B scaffold inhibitor.

12. A kit comprising
(a) a Sin3B Complex inhibitor; and
(b) a chemotherapeutic or radiotherapeutic agent.


SEQUENCE LISTING (7 sequences)

SEQ ID NO: 1 - protein, 20 aa, Artificial sequence
  Synthetic peptide
VRMNIQMLLEAADYLERRER

SEQ ID NO: 2 - protein, 14 aa, Artificial sequence
  Synthetic peptide
MNIQMLLEAADYLE

SEQ ID NO: 3 - protein, 14 aa, Artificial sequence
  Synthetic peptide
MNIQMLLEAPDYLE

SEQ ID NO: 4 - protein, 14 aa, Artificial sequence
  Synthetic peptide
MNIQMPLEAPDYLE

SEQ ID NO: 5 - protein, 11 aa, Artificial sequence
  Synthetic peptide
YGRKKRRQGGG

SEQ ID NO: 6 - RNA, 21 nt, Artificial sequence
  Synthetic oligonucleotide
gcaaagcggucccuguuuauu

SEQ ID NO: 7 - RNA, 21 nt, Artificial sequence
  Synthetic oligonucleotide
ggcaaugggucgugcgagauu